(19) United States
(12) Patent Application Publication
(10) Pub. No.: US 2002/0019332 A1
(43) Pub. Date: Feb. 14, 2002

(54) METHOD OF INDUCING APOPTOSIS IN B-LYMPHOCYTES

(76) Inventors: JOHN G. MONROE, PHILADELPHIA, PA (US); LESLIE B. KING, ARDMORE, PA (US)

Correspondence Address: JANE MASSEY LICATA, 66 E MAIN STREET, MARLTON, NJ, 08053

(21) Appl. No.: 09/347,334

(22) Filed: Jul. 2, 1999

(51) Int. Cl. A01N 37/18; A61K 38/00; A61K 39/00; A61K 39/38

(52) U.S. Cl. 514/2; 424/184.1

(57) ABSTRACT

A method for inducing apoptosis in B-lymphocytes is provided that involves contacting B lymphocytes with an inhibitor of protein kinase C.

### INTRODUCTION

This invention was supported in part by funds from the U.S. government (NIH Grant No. RO132) and the U.S. government may therefore have certain rights in the invention.

### BACKGROUND OF THE INVENTION

B-lymphocyte-mediated immune reactions impede treatment following transplantation or gene therapy and are central to the development of autoimmune disease. During autoimmune disease development, B-lymphocytes become acutely or chronically activated to proliferate and secrete antibodies reactive with self tissues and/or serum and interstitial proteins. In the case of allogenic and xenogenic transplantation, B-lymphocytes reactive to non-cross reactive antigens on the transplanted tissue are activated to secrete antibodies that are central to or participate with other components of the immune system to reject transplanted tissue. In particular, rejection of xenografts occurs almost entirely due to B-lymphocyte reactions to transplanted tissue. In addition, a complicating factor in establishing an effective approach to viral-based gene therapy delivery is the immune response to the viral vectors as well as to the therapeutic proteins whose expression is driven by these vectors. B-lymphocyte-mediated immune responses are central in many instances where gene therapy fails. Current treatment to modulate the B lymphocyte immune reaction in each of these conditions has relied on general immunosuppression of either the activation or effector function of B-lymphocytes. Such treatment generally leaves the patient immunocompromised and can lead to development of opportunistic infections and malignancies, conditions that can be life-threatening. Therefore, there is a need for more selective approaches to reducing B-lymphocyte activation in conditions such as autoimmunity, transplantation and gene therapy, which are associated with and characterized by reactive B-lymphocytes.

Throughout B cell development, signals transduced through the B cell antigen receptor (BCR) play an important role in regulating B cell maturation (Basten, A. R. et al. 1991. Immunol. Rev. 122:5; Nemazee, D. et al. 1991. Immunol. Rev. 122:117). However, BCR-iduced signals can lead to dramatically different functional responses, depending on the maturational stage of the B cell. For instance, although both immature and mature B cells express the mature antigen-binding form of the BCR, immature B cells undergo negative selection, or are tolerized, in response to receptor ligation, while mature B cells are induced to proliferate and secrete immunoglobulin (Ig). Immature B cells in the bone marrow that have just begun to express surface IgM, as well as late-immature or transitional-stage B cells that have recently emigrated from the bone marrow to the spleen and that express high levels of surface IgM and low levels of surface IgD, are sensitive to this tolerization process (Carsetti, R. et al. 1995. J. Exp. Med. 181:2129; Allman, D. M. et al. 1992. J. Immunol. 149:2533; Allman, D. M. et al. 1993. J. Immunol. 151:4431). The sensitivity of immature B cells to tolerization following antigenic exposure is thought to be critical for the maintenance of immunological self-tolerance. Immature B cells are thought to be tolerized by a number of mechanisms including clonal anergy (Goodnow, C. C. et al. 1988. Nature 334:676), receptor editing (Gay, D. et al. 1993. J. Exp. Med. 177:999), competition for follicular niches (Cyster, J. G. et al. 1994. Nature 371:389) and clonal deletion (Hartley, S. B. et al. 1993. Cell 72:325). The number of newly generated immature B cells greatly exceeds that of mature B cells, suggesting that a vast majority of these cells undergo antigen-mediated negative selection. Although the relative susceptibility of immature B cells to tolerance induction has been appreciated for many years, the molecular events that distinguish an activation signal in mature cells from a tolerogenic signal in immature cells has yet to be elucidated. Therefore, it has not been possible to exploit these events to produce a decrease in the number of reactive B-lymphocytes.

The possible mechanisms investigated have included a role for T cells, specifically an absence of T cell activity (Bretscher, P. A. and M. Cohn. 1970. Science 189:1042) and differential signaling through surface IgM and surface IgD (Carsetti, R. et al. 1993. Eur. J. Immunol. 23:168). However, experiments have failed to definitively link these events to B cell tolerization. Ligation of IgD on immature, transitional B cells induces apoptosis and fails to protect the cells from anti-IgM-induced apoptosis (Norvell, A. and J. G. Monroe. 1996. J. Immunol. 156:1328), indicating that the differential responses of immature and mature B cells cannot be attributed to a protective signaling mechanism transduced through surface IgD. These results are consistent with studies in transgenic mice that express either anti-hen egg lysozyme IgM or IgD where exposure to antigen led to efficient tolerization in both IgM-only and IgD-only expressing mice. The data suggest that differential sensitivity to negative selection of the immature and mature B-lymphocyte must be due to intrinsic, developmentally regulated differences in antigen-mediated signal transduction pathways.

Antigen receptor ligation of mature B cells leads to activation of multiple signaling pathways, including those involving tyrosine kinases and phosphatases, Ras and mitogen-activated protein kinases (MAPKs) and phosphoinositide-3 kinase (Cambier, J. C. et al. 1994. Ann. Rev. Immunol. 12:457). Activation of the src family kinases following BCR engagement leads to the eventual tyrosine phosphorylation and subsequent activation of phospholipase Cγ, resulting in the breakdown of phosphoinositide bis phosphate (PIP2) into the second messengers inositol triphosphate (IP3) and diacylglycerol (DAG). IP3 is thought to be at least partially responsible for the release of calcium from intracellular stores while DAG can activate a subset of protein kinase C (PKC) isoenzymes, including PKC-α, PKC-β, PKC-γ, PKC-δ, and PKC-ε. It is not known which of the isoenzymes is involved and to what extent. It has been demonstrated that PKC-β-deficient mice exhibit a phenotype that is similar to the xid immunodeficient mouse strain (Leitges, M. et al. 1996. Science 273:788), and although they presumably express other PKC isoenzymes, they appear to be unable to replace PKC-β signaling through the BCR, demonstrating a unique requirement for this isoenzyme in B-lymphocyte activation.

It has now been found that activation of PKC isoenzymes can rescue immature B-lymphocytes from BCR-induced apoptosis. In addition, mature B-lymphocytes that are either depleted of PKC or are stimulated in the presence of PKC inhibitors are susceptible to BCR-induced apoptosis, so that uncoupling of PKC from BCR-induced signal transduction leads to negative selection of developing B-lymphocytes.

### SUMMARY OF THE INVENTION

An object of the present invention is a method for inducing apoptosis in B-lymphoctyes which comprises contacting B-lymphocytes with an inhibitor of protein kinase C.

Another object of the present invention is a method of treating a condition in an animal that is associated with or characterized by increased activity of B-lymphocytes which comprises administering to an animal suffering from such a condition an inhibitor of protein kinase C so that B-lymphocyte activity is reduced. Such conditions include autoimmunity, transplantation and associated post-transplantation tissue rejection, and gene therapy.

### DETAILED DESCRIPTION OF THE INVENTION

A method for inducing apoptosis in B-lymphocytes has been developed that is based on inhibition of PKC activity. The ability to induce apoptosis in this population of immune cells leads to decreased activity of B-lymphocytes and improvement in conditions associated with or characterized by an increased activity of these immune cells. Examples of such conditions include but not be limited to autoimmunity, post-transplantation tissue or organ rejection and gene therapy.

It is known that mature B-lymphocytes proliferate in response to BCR engagement while immature and transitional B-lymphocytes undergo apoptosis in response to anti-Ig stimulation and are sensitive to negative selection. This difference in functional phenotype is associated with a unique intracellular signaling response in each cell type. Mature B-lymphocytes increase PIP2 hydrolysis and elevate intracellular calcium levels in response to surface Ig crosslinking while immature B-lymphocytes increase intracellular calcium levels in the relative absence of increased PIP2 hydrolysis (Yellen, A. J. et al. 1991. J. Immunol. 146:1446). Since PIP2 hydrolysis is known to lead to PKC activation, studies were performed to investigate whether BCR-induced signal transduction events that were uncoupled from PKC activation would lead to negative selection.

Experiments were performed using an in vitro model system that makes use of an immature population of B cells purified from the spleens of adult mice after sublethal irradiation. Immature B cells obtained from these autoreconstituting mice are phenotypically immature and respond identically to immature B cells purified from either human neonatal spleen or adult human bone marrow (Norvell, A. et al. 1995. J. Immunol. 154:4404; Norvell, A. and J. G. Monroe. 1996. J. Immunol. 156:1328). This homogeneous population of immature B cells is devoid of pre-B cells and mature B cells found in neonatal spleen and adult bone marrow, respectively. Further, unlike the immature B cells purified from either bone marrow or neonatal spleen, immature B cells from autoreconstituting mice can be isolated without positive selection on anti-Ig panning plates. Since large numbers of unmanipulated immature B cells can be obtained, autoreconstituting B cells are an ideal system for analyzing biochemical events responsible for tolerogenic signal transduction. The B cells isolated from these mice represent a transitional stage of development in which cells are subject to negative selection. This in vitro system uses apoptosis as an indicator of negative selection.

Purified populations of either immature B cells obtained from the spleens of day 14 autoreconstituting mice or mature B cells isolated from spleens of normal adult mice were incubated in the absence or presence of 50 μg/ml of rabbit anti-mouse IgM F(ab′)2 fragments (anti-Ig) for 16-18 hours. Addition of anti-Ig to immature B cells resulted in a substantial increase in the frequency of apoptotic cells as determined by the increased number of cells containing subdiploid amounts of DNA as detected by flow cytometry. Similar results were observed using a modified TUNEL technique that detects nicked DNA. In contrast, overnight culture of mature splenic B cells in the presence of anti-Ig resulted in only a marginal increase in the frequency of apoptotic cells over that observed in unstimulated cultures. These results confirmed that in the absence of T cells assistance, immature and mature B cells respond differentially to BCR signaling and that immature B cells are susceptible to BCR-induced apoptosis.

Experiments were also performed to determine if the inability to activate PKC in response to BCR-cross-linking was directly related to the induction of apoptosis, where pharmacologic activation of PKC in immature B cells may protect from anti-Ig-induced death. Isolated immature B cells were cultured with the phorbol ester, PMA, which bypasses the antigen receptor and activates DAG-dependent PKC isoenzymes directly. Late-stage immature B cells were cultured for 18 hours in the presence of medium alone, anti-IgM F(ab′)2 fragments (50 μg/ml), PMA (10 ng/ml) or the combination of anti-Ig and PMA. After culture, the cells were harvested and assayed for relative DNA content. Activation of PKC in the immature B cell population resulted in significant protection from anti-IgM-induced apoptosis. The ability of phorbol esters to activate PKC was critical for the observed response because in contrast to the biologically active stereoisomer 4αPDD which efficiently rescued cells, the inactive stereoisomer 4βPDD did not rescue cells. This demonstrated that while BCR-induced signaling events can lead to cell death, activation of PKC by itself was sufficient to rescue cells from apoptosis.

Results showing the relative inability of immature B cells to increase PIP2 hydrolysis in response to BCR ligation as well as results showing the ability of PMA to rescue immature B cells from BCR-induced apoptosis indicated that BCR-induced activation of PKC was necessary to prevent anti-Ig induced death. Therefore, experiments were performed to determine if PKC-depleted mature B cells were susceptible to BCR-induced apoptosis. The phorbol ester PDBu was used because it is effective in depleting PKC yet it is less hydrophobic than PMA and can therefore be more easily removed from cultures by washing. PKC depletion in PDBu- or mock-treated cultures was monitored by Western blot analysis. The two conventional phorbol ester-responsive isoenzymes of PKC expressed in B cells (PKCα and PKCβ) are efficiently depleted in PDBu-treated cells while the two novel isoenzymes (PKCδ and PKCε) are also depleted, although not as efficiently. The level of expression of the lower molecular weight phorbol-ester insensitive PKCζ was decreased only slightly, as expected. PKC levels in unstimulated immature and mature cells were quite similar, making it unlikely that differential regulation of PKC activity would occur at the level of expression of the individual isoenzyes.

To assess the role of PKC activation in the prevention of antigen-mediated apoptosis, the ability of anti-Ig to induce apoptosis in PKC-depleted mature B cells was determined. Mature-stage B cells were cultured overnight with or without 100 ng/ml of PDBu. After overnight treatment, the cells were washed expansively and subjected to an apoptotis analysis in which B cells were recultured with medium alone, anti-Ig, anti-Ig and PMA or anti-Ig and 4αPDD. After 8 hours incubation, the cells were harvested and subjected to a modified TUNEL analysis. While mock-depleted B cells did not show increases in the percentage of apoptotic cells in any of the culture conditions, there was a significant and striking increase in the number of apoptotic cells with PDBu treatment. After PKC depletion, mature B cells were specifically susceptible to anti-Ig-mediated apoptosis. These data demonstrated that an uncoupling of PKC activation from BCR-induced signal transduction was sufficient to render mature B cells insensitive to anti-Ig-induced apoptosis. PMA was still capable of rescuing the cells from anti-Ig-induced apoptosis.

The role of PKC in preventing BCR-induced apoptosis of immature B cells was also examined using inhibitors of PKC. Addition of two specific PKC inhibitors, bisindoylmaleimide I, which inhibits both conventional and novel PKC isoenzymes, and Ro-32-0432, an inhibitor with a greater selectivity for conventional isoenzymes, 15 minutes before stimulation of mature B cells with anti-Ig, led to induction of apoptosis. This effect of enhanced cell death was not due to toxic effects of the inhibitors because the frequency of apoptosis in unstimulated or LPS-stimulated mature B cells was not affected.

The ability of PMA to protect immature B cells from BCR-induced apoptosis was assessed in the presence of the same two inhibitors. Prior addition of PKC inhibitors substantially blocked the PMA-induced protection of immature B cells from BCR-induced apoptosis. Again, these inhibitors are relatively non-toxic, as the frequency of apoptotic cells in unstimulated or LPS-stimulated immature B cells was unaffected. Therefore, these data show that PKC inhibitors of various types are able to render mature B cells susceptible to apoptosis without producing significant toxicity. The fact that both selective and non-selective inhibitors were effective indicates that this response is not dependent on one isoenzyme form alone.

Since PMA was capable of rescuing mature, PKC-depleted B cells from anti-Ig-induced apoptosis, experiments were performed to assess whether PMA was capable of desensitizing the BCR in these cells. While PMA efficiently reduced the BCR-induced increase in intracellular calcium in mock-treated B cells, it did not alter the BCR-induced calcium flux in PDBu-treated cells at all, as measured in Fura-2-AM loaded cells. These results demonstrate that the ability of PMA to rescue B cells from BCR-induced apoptosis is not the result of receptor desensitization.

These data also demonstrated that cross-linking the antigen receptor of immature B cells initiates intracellular biochemical changes. The induction of apoptosis is not associated with the absence of signal transduction in immature B cells but rather is an active process that is initiated by BCR-cross-linking and subsequent signaling events. The ability of PMA treatment to inhibit anti-Ig mediated apoptosis as well as the sensitivity of PKC-depleted mature splenic B cells to anti-Ig apoptosis supports the role of PKC activation in protection from anti-Ig-induced apoptosis. Contact of B cells with specific inhibitors of PKC has been shown to be an effective method to induce apoptosis in the B cells and thus reduce the number of B cells available for activity.

In a preferred embodiment, inhibition of the activity of PKC is achieved by administering to an animal, including humans, a PKC inhibitor compound. The compound converts an immune response into a response leading to apoptosis and deletion of antigen-reactive B-lymphocytes from the circulating B-lymphocyte population. Such treatment is selective for B-lymphocytes that are reactive to antigen and spares the remainder of the B-lymphocytes. This is an important advantage for this method over currently available methods of treatment of conditions associated with activation of B-lymphocytes which include, but are not limited to, autoimmunity, transplantation (i.e., post-transplantation tissue rejection) and gene therapy, which involve general immunosuppression that affects activated and non-activated B cell populations. The method of the present invention involves only a short treatment period. It has now been shown that the apoptotic response is triggered within 12-16 hours after inhibition of PKC. This targeted and rapid treatment rids the animal of auto-reactive and active B-lymphocytes in a short window of immunosuppression. One of skill would understand how to administer PKC inhibitors as well as how to choose doses for a PKC inhibitor based on knowledge generally available in the art and the results of the in vitro studies presented. Doses for use in in vivo studies are commonly developed based on results in cells in culture using fundamental principles of pharmacology which dictate extrapolation from such data. The PKC inhibitor would be administered to the animal by a variety of routes including but not limited to intravenous, subcutaneous and intramuscular injection in a pharmaceutically acceptable carrier or diluent. Any PKC inhibitor could be used that has been shown to have activity in vitro to inhibit PKC without significant toxicity, as shown in the instant invention. Examples of such inhibitors are known in the art and include but are not limited to, melittin, CGP-41251, calphostin C, H7, staurosporino, vincristino, daunomycin, paclitaxel, tamoxifin, RO 31-7549 and bryostatin. Information on dosing and routes of administration for these compounds is known in the art.

The following non-limiting examples are provided to further illustrate the invention:

### EXAMPLES

### Example 1

### Antoreconstituting Mice

Adults BALB/c mice 8-10 weeks of age were subjected to 500 rad of whole body γ radiation. The peripheral lymphoid compartments were allowed to reconstitute over a period of 13-15 days. F(ab′)2 fragments of polyclonal rabbit anti-mouse IgM were created (Monroe, J. G. and M. J. Kass. 1985. J. Immunol. 135:1674).

### Example 2

### B-Lymphocyte Purification

Splenic B cells were prepared (Norvell et al. 1995. J. Immunol. 154:4404). Adult mice were killed by cervical dislocation at 8-10 weeks of age. Spleens were removed aseptically and placed in HBSS+2% FCS. Single cell suspensions were prepared by grinding spleens between the frosted ends of glass slides. To remove T cells, the cells were treated with anti-Thy 1.2 (HO-13-4), rabbit complement and DNase I. Red blood cells were lysed by treatment with Gey's solution and the cells were size fractionated by passage over a 50/75% Percoll gradient. B cells from autoreconstituting mice were isolated in an analogous manner. In autoreconstituting mice, B cell populations are regenerated in a relatively synchronous wave. Late-stage immature or transitional-stage B cells can be found in the spleens of autoreconstituting mice as early as day 13 and persist as long as day 18. After this time, the cells begin to acquire the phenotypic characteristics of mature B cells and after day 21 they respond functionally as mature B cells.

### Example 3

### Apoptosis Assay

Purified B cells were cultured under various conditions overnight in either sterile 2 ml click-cap tubes or 96-well plates. PKC inhibitors were added 15 minutes before the addition of the stimulant. After 14-18 hours in the case of immature B cells or 24 hours in the case of mature B cells, the cells were harvested and washed in PBS containing 2% FCS. Cells were fixed and permeabilized by resuspending in 70% ethanol made in 1xPBS and incubating at −20° C. for at least 2 hours. After incubation, the cells were washed twice with PBS +2% FCS and finally resuspended in a staining solution made in 1xPBS containing 50 μg/ml of RNase A, 0.01% sodium azide, and 10 μg/ml of propidium iodide. The cells were stained at 4° C. overnight and analyzed by flow cytometry on a Becton Dickinson FACScan using LYSYS II software. The mean percentages of apoptotic cells and the standard errors for each culture condition were determined and significance was assessed by the Student's t-test.

### Example 4

### Measurement of Intracellular Calcium

Purified B cells were washed in HBSS without phenol red and supplemented with 2% FCS. For loading, cells were resuspended in HBSS without phenol red and supplemented with 10% FCS. Fura-2 acetoxymethyl ester was added to a final concentration of 1 μM from a 1 mM stock made in DMSO. Cells were incubated in light tight tubes for 30 minutes at 30° C. with occasional inversion. Cells were then washed twice with HEBSS+2% FCS and resuspended at a concentration of 3.3×10⁷/ml in HBSS+10% FCS. Fluorometric analysis of dyed cells was performed using a luminescence spectrometer. Excitation was at 340 nM and 380 nM with emission monitored at 510 nM. B cells were diluted to 2×10⁶/ml in HBSS and equilibrated for 2 minutes, at which time a baseline spectrum was read. The cells were stimulated with rabbit anti-mouse IgM F(ab′)2 fragments (50 μg/ml) and the response measured. Cells were then lysed with Triton X-100 and Fₘₐₓ and Fₘᵢₙ were measured by the chelation of extracellular calcium with the addition of EGTA. Calcium traces were calculated by the Intracellular Biochemistry Application (Perkin-Elmer) using these values.

### Example 5

### Western Blot Analysis

For analysis of postnuclear extracts, 1×10⁷ cells were lysed in 50 μl of Triton-X lysis buffer (0.1% Triton-X-100; 100 mM Tris, pH 8.0; 10.8 μg/ml aprotinin; 1.5 μg/ml each of leupeptin, pepstatin A, chymostatin, and antipain; 2 mM phenylmethylsulfonyl fluoride; 1 mM N-ethylmaleimide) on ice for 30 minutes. Lysates were microcentrifuged at 14,000× g at 4° C. for 10 minutes to remove the Triton-X insoluble fraction. Lysates from equivalent cell numbers were fractionated on SDS-PAGE gels and electroblotted onto Immobilon (Millipore, Bedford, Mass.). Blots were incubated overnight in Tris-buffered saline with Tween-TBST (10 mM Tris, pH 8.0; 137 mM NaCl; 0.05% Tween 20) plus 2% bovine serum albumin to block nonspecific binding. Membranes were washed twice with TBST and probed with the appropriate antibody solutions for 90 minutes, then washed 3 times in TBST, followed by secondary incubation with the appropriate horseradish peroxidase-conjugated reagents. Three washes with TBST were conducted before detection with an enhanced chemiluminescence system.

### Example 6

### Modified TUNEL Assay

In some cases, the frequency of apoptotic cells was quantitated using a modified TUNEL assay. Briefly, 1×10⁶ cells were fixed in 2.5% formaldehyde in 1xPBS. The fixed cells were permeabilized in 0.2 ml 0.1% Triton/0.1% citrate for 2 minutes on ice. After washing, cells were incubated in 50 μl of nick translation reagent, which consists of 5 μl nick translation buffer (500 mM Tris, pH 7.5; 100 mM MgSO4; 1 mM DTT), 0.03 μl of fluorescein-dUTP (1 nmol/μl), 1 82 l dTTP (0.7 nmol/μl), 1 μl each of dATP, dCTP, dGTP (1 nmol/μl) and 0.1 μl DNA polymerase (5 U/μl) and incubated for at least 1 hour at 37° C. Cells were then washed and analyzed by flow cytometry.

What is claimed is:

1. A method of inducing apoptosis in B-lymphocytes comprising contacting B-lymphocytes with an inhibitor of protein kinase C.

2. A method of treating a condition in an animal characterized by increased activity of B-lymphocytes comprising administering to an animal an inhibitor of protein kinase C so that B-lymphocyte activity is reduced.
